(19) United States
(12) Patent Application Publication
(10) Pub. No.: US 2006/0002865 A1
(43) Pub. Date: Jan. 5, 2006

(54) Stable baking soda/peroxide with calcium and phosphate whitening product

(76) Inventor: Adonis Buelo, Leonardo, NJ (US)

Correspondence Address: Stephen B. Shear;Church & Dwight Co., Inc.- Law Dept., 469 North Harrison Street, Princeton, NJ, 08540, US

(21) Appl. No.: 10/882,063

(22) Filed: Jun. 30, 2004

(51) Int. Cl. A61K 7/20

(52) U.S. Cl. 424/53

(57) ABSTRACT

A dentifrice containing sodium bicarbonate, a peroxide generator, and remineralizing calcium and phosphate salts is provided within an anhydrous vehicle to maintain stability and palatability.

### FIELD OF THE INVENTION

The present invention is directed to stable toothpastes which contain baking soda and peroxide and have whitening and remineralization ability.

### BACKGROUND OF THE INVENTION

The use of bicarbonate salts (baking soda) as a dentifrice or the incorporation of such salts into dentifrice compositions is well known in the art of oral care. A renewed interest in incorporating bicarbonate salts into toothpaste has emerged in light of the success of the present assignee's Dental Care® and PeroxiCare® products. The addition of bicarbonate salts into dentifrices is beneficial for several reasons, including its plaque removing capabilities, as well as its ability to enhance the whitening properties of dentifrices. More importantly, bicarbonate salts provide a clean fresh feeling in the oral cavity after brushing and rinsing with water.

It has been demonstrated that combinations of various salts and hydrogen peroxide solution, when properly applied as part of a treatment under the supervision of a dentist, are effective in controlling periodontitis. B. G. Rosling et al., Journal of Clinical Periodontology, Vol.10 pp. 487-514, 1983. Sodium bicarbonate, a particularly convenient and palatable non-toxic salt, is believed to be effective in this treatment. Keyes P. H. et al, Quintessence International No. 1, January 1978, report 1590, pp. 51-56 and No. 2, February 1978, pp. 69-75.

The bacteria causing periodontal disease are anaerobic. Armitage, G. C., Biological Basis of Periodontal Maintenance Therapy, 1980. By providing high levels of oxygen, hydrogen peroxide is believed to be effective in killing these bacterial organisms. Hydrogen peroxide is the preferred oxidizing agent as it is readily available, and has been proven effective and non-toxic. Various dentifrices have been formulated which contain oxidizing agents such as sodium perborate (Cella, et al., U.S. Pat. No. 3,885,028 and Molnar, U.S. Pat. No. 2,275,979), potassium chlorate, urea peroxide (Gordon, U.S. Pat. No. 4,522,805 and Schaeffer, U.S. Pat. No. 4,528,180) and magnesium peroxide. Balsam, M. S. et al, Cosmetics: Science and Technology, Volume 1, Second Edition, Wiley Interscience (1972) page 496.

Sodium perborate and potassium chlorate do not release significant levels of hydrogen peroxide in water. Kirk-Othmer Encyclopedia of Chemical Technology, Third Edition, Volume 17, pages 1-22; Kern, D. M., J. Am. Chem. Soc. 77:5458, 1955. Although sodium perborate has been classified category 1 (Federal Register, Oct. 7, 1982) for temporary use as an oral wound cleaner, it is of questionable safety for frequent topical use on the mucous membranes of the mouth and throat because it contains boron which can undergo systemic absorption. (Federal Register Vol. 44 No. 214 page 63282, Fri., Nov. 2, 1979, Proposed Rules). Sodium perborate also has an undesirably low solubility in water of about 2.5%. This low solubility limits the concentration of oxidizing agent. Magnesium peroxide, an essentially insoluble salt in water, is similarly undesirable. Handbook of Chemistry and Physics, 59th Ed., 1978-79.

Various peroxide releasing dentifrice formulations utilize urea peroxide as the oxidizing agent. See U.S. Pat. No. 4,522,805. However, sodium bicarbonate/urea peroxide dentifrices are not stable, presumably because hydrogen peroxide solutions are not stable at alkaline pH (see “Hydrogen Peroxide,” Schumb, Satterfield & Wentworth, American Chemical Society Monograph No.128 (1955), pp. 526-530).

Oral compositions containing both a peroxide and sodium bicarbonate have been acclaimed by the dental profession, especially through the work of Keyes. See Keyes et al “Periodontics and Oral Hygiene,” January 1978, pages 51-56. Unfortunately, formulations based on the Keyes technology are particularly prone to decomposition.

Several approaches have been reported to overcome difficulties presented by such combination of ingredients. U.S. Pat. No. 3,577,521 (Scheller) discloses use of acid salts, e.g., phosphate salts, in combination with alcohol-silica gels to obtain a foaming storage-stable toothpaste of peroxide-bicarbonate.

U.S. Pat. No. 4,837,008 (Rudy et al) overcomes the problem through a non-aqueous dentifrice wherein an inorganic peroxide and/or bicarbonate is coated with a water-soluble barrier which is insoluble in the dentifrice vehicle.

Physical isolation of the peroxide from co-reactive ingredients into separate compartments has been another approach to the problem. U.S. Pat. No. 4,849,213 and U.S. Pat. No. 4,528,180, both to Schaeffer, disclose a dual-compartment package containing respective peroxide gel and bicarbonate paste components. U.S. Pat. No. 4,487,757 (Kiozpeoplou) discloses a toothpaste that physically segregates sodium bicarbonate from acidic ingredients to prevent contact therebetween prior to usage.

Alkali and alkaline earth metal percarbonates, e.g., ammonium percarbonate, were also described as peroxide releasing agents for dentifrices almost 100 years ago. (Gane, U.S. Pat. No. 802,099 granted Oct. 17, 1905.) A safe, palatable and convenient formulation containing sodium bicarbonate and sodium percarbonate in the form of a stable tooth powder has been described in commonly assigned U.S. Pat. No. 4,812,308, issued Mar. 14, 1989. Sodium carbonate forms a crystalline addition compound with hydrogen peroxide, corresponding to the formula 2 Na₂CO₃.3H₂O₂ (sodium carbonate sesquiperoxide), and is commonly known as sodium percarbonate. The compound has many potential applications as a peroxygen source when dissolved in an aqueous medium. Sodium percarbonate has a high active oxygen content (15.28% theoretical) and high water solubility. It is produced from low cost starting materials, and it is an environmentally safe chemical. Stable toothpaste or gel dentifrices are currently known which combine sodium bicarbonate with sodium percarbonate. Such products are disclosed in commonly assigned U.S. Pat. Nos. 4,891,211; 5,374,368; 5,424,060; and 5,496,542.

It has been found that toothpastes or gels containing a mixture of sodium bicarbonate and sodium percarbonate in a carrier free of glycerin and which contains polyethylene glycol, see U.S. Pat. No. 4,891,211, either with or without other dentifrice adjuvants such as fluorides (e.g., sodium fluoride), sweeteners (e.g., saccharin), flavorants, etc., are quite palatable and surprisingly stable with regard to loss of oxygen, as compared with dentifrices prepared from other bicarbonate/hydrogen peroxide-releasing formulations, e.g., those containing urea peroxide.

The primary component of the enamel and dentin in teeth is calcium phosphate in the form of calcium hydroxyapatite. While highly insoluble at normal oral pHs, the calcium phosphate in the teeth tends to be relatively soluble in acidic media. Consequently, carious lesions can form in the subsurface of a tooth when such tooth is exposed to acids formed from the glycolysis of sugars caused by various oral bacteria.

Because saliva is supersaturated with respect to calcium and phosphate ions, saliva helps protect teeth against demineralization and can slowly remineralize teeth which have become demineralized by acids. It is well known that fluoride ions can enhance the natural remineralization process and this is one of the accepted mechanisms by which fluoride toothpastes and rinses protect against caries. However, the efficacy of fluoride-containing toothpastes and rinses to remineralize teeth is limited by the modest levels of calcium and phosphate in saliva. It is highly desirable to increase the available concentration of calcium and phosphate ions in the oral cavity to speed up the remineralization process. However, because of calcium phosphate's low solubility at the pH of saliva, the addition of higher levels of dissolved calcium and phosphate ions is not easily accomplished.

Remineralizing/mineralizing one-part and two-part oral products are disclosed in commonly assigned U.S. Pat. Nos. 5,603,922; 5,605,675; and 5,571,502. Each of these patents is herein incorporated by reference in its entirety.

U.S. Pat. No. 5,603,922 discloses one-part and two-part products and methods of using same to remineralize subsurface lesions. The one-part and two-part products contain at least one water-soluble calcium salt, at least one water-soluble divalent metal salt wherein the divalent metal is other than calcium and at least one water-soluble phosphate salt. In the two-part products, the calcium and divalent metal salts are disposed in a first discrete component, and the phosphate salt(s) is disposed in a second discrete component. The two-part product may further contain a dispensing means for allowing the first and second components to be simultaneously dispensed from the product so as to permit the dispensed first and second components to simultaneously contact the tooth or teeth being treated. The aqueous solution formed by mixing the salts used in the one-part and two-part products has a pH of from about 4.0 to about 7.0.

U.S. Pat. No. 5,605,675 discloses a two-part product and method of using same for remineralizing dental enamel, wherein the product contains a first discrete component containing at least one water-soluble calcium salt and a second discrete component containing at least one water-soluble phosphate salt and at least one water-soluble fluoride salt. The product may further contain a means for allowing the first and second components to be simultaneously dispensed from the product. The first and second components of the product each have a pH such that when the two components are mixed to form an aqueous mixed solution, the solution has a pH of from about 4.5 and 10.0.

U.S. Pat. No. 5,571,502 is directed to one-part, non-aqueous products and methods of using same for remineralizing subsurface lesions, wherein the products contain at least one water-soluble calcium salt; at least one water-soluble phosphate salt; either a stabilizer or a hydrophilic, non-aqueous, water-soluble vehicle; and, optionally, at least one water-soluble fluoride salt. When the components are mixed with water or saliva to form an aqueous mixed solution, the solution has a pH of from about 4.5 to about 10.0.

In the one-part and two-part products disclosed in the foregoing applications, the cationic and anionic components are kept separate from one another until use of the product. In addition, the cations and anions are delivered simultaneously to the surface of the tooth being treated. These factors, along with the pH of the aqueous solution and the use in some cases of at least one water-soluble divalent metal salt, are helpful in allowing the cations and anions to have ample time to diffuse through the surface of the tooth to the subsurface before undergoing precipitation.

For mineralization or remineralization of enamel or dentin to occur, the concentration of calcium and phosphate ions in saliva must be above the concentration required to saturate the solution with respect to the formation of calcium hydroxyapatite, octacalcium phosphate, dicalcium phosphate dihydrate, or other form of insoluble calcium phosphate. At pHs above about 6.5, these requirements are met by the levels of calcium and phosphate in normal human saliva. However, because the concentration of calcium and phosphate ions in normal human saliva is only modest, even at pHs above 6.5, the rate of mineralization produced by normal saliva is very slow even when fluoride is present to catalyze the process. When the pH is above about 7, raising the concentration of calcium and phosphate ions much beyond that normally present in saliva does not, however, significantly increase remineralization. Because of the high insolubility of calcium phosphate salts above pHs of about 7, excessively rapid precipitation occurs which does not allow time for the ions to penetrate the tooth.

At pHs below about 7, significant remineralization will occur only if the concentration of calcium and phosphate ions in the saliva is above the concentration required to saturate the solution with respect to the formation of dicalcium phosphate dihydrate. Under these pH conditions, it has been demonstrated that remineralization can be accelerated by increasing the degree of supersaturation in saliva. Inasmuch as the solubility of dicalcium phosphate increases with decreasing pH, it has been found that when lesions are remineralized with solutions having a pH in the range of 4.5 to 7.0 and containing supersaturated quantities of calcium and phosphate ions, the optimum concentration of calcium ions needed to maximize the process increases with decreasing pH. Below a pH of about 4.0, dicalcium phosphate dihydrate becomes the stable precipitating species from supersaturated solutions. Under these pH conditions, it takes very high levels of calcium and phosphate to saturate the solutions. Under such conditions, there is a real danger with fairly high concentrations of calcium and phosphate that the solution will be undersaturated and demineralization of the teeth being treated will occur.

It has also been found that the simultaneous provision of very high calcium and phosphate ion concentrations can result in premature precipitation of the calcium salt before the ions can penetrate the tooth or premature precipitation so as to block the entrances of the pores in tooth enamel and dentin and thereby prevent further remineralization.

Thus, a problem apparently exists in that to control untimely precipitation, the concentration of either the dissolved calcium ions or the dissolved phosphate ions needs to be limited. This in turn would be expected to disadvantageously limit the maximum rate of mineralization or remineralization which could be accomplished.

In addition, the use of very high calcium and fluoride ion concentrations can result in premature precipitation of the fluoride ions before these ions can penetrate the tooth. As mentioned previously herein, fluoride ions can enhance the natural remineralization process. However, sufficient levels of fluoride ions are generally required to be present. Calcium cations and fluoride anions precipitate to form calcium fluoride, a salt which is sparingly soluble in water. The formation of calcium fluoride is undesirable since it reduces the amount of free fluoride anions available for use in the remineralization process. Thus, it is desirable to provide a remineralizing product wherein the solution used to treat the teeth contains sufficient levels of dissolved fluoride anions to enhance the remineralization of the subsurface lesions.

As disclosed in commonly assigned U.S. Pat. No. 6,159,448, the delayed precipitation of calcium cations and phosphate and fluoride anions until such ions have diffused through the tooth surface to the subsurface and/or dentin can be achieved by using at least one calcium salt having partial water-solubility at a pH of from about 4.0 to about 10.0. With the use of the partially water-soluble calcium salt(s), the calcium cations and the phosphate and fluoride anions in the mixed aqueous composition used to treat the tooth are able to remain soluble for the period of time sufficient to allow the cations and anions to diffuse through the surface of the tooth to the subsurface and/or dentin thereof, where, as stated above, the ions react to form an insoluble precipitate on the demineralized lesion(s) and/or exposed tubule(s). Moreover, the use of at least one partially water-soluble calcium salt allows a greater level of free fluoride anions to be available for absorption by the tooth being treated than would be available when water-soluble calcium salts are used instead. Thus, an important advantage of using the partially water-soluble calcium salt(s) is that at any point in time the low concentration of calcium cations does not insolubilize either the phosphate anions or the fluoride anions, the cations and anions both being used in the remineralization and/or mineralization process. Calcium sulfate and various hydrates thereof are particularly useful. The entire content of U.S. Pat. No. 6,159,448 is herein incorporated by reference.

It would be quite useful to provide an oral product such as a toothpaste which contained the remineralizing salts of calcium, phosphate, and fluoride, and also contained a peroxide-generating component for whitening and bactericidal properties for preventing periodontal disease, along with a bicarbonate salt for whitening and for providing a fresh mouth feel. Unfortunately, in view of the discussion above, providing a stable composition which contains all the above components is problematic in as much as the calcium salts readily react with the phosphate and fluoride salts, and as well, the bicarbonate and peroxide-generating compounds often react prematurely prior to use in the oral cavity.

### SUMMARY OF THE INVENTION

In accordance with the present invention, a stable oral product in the form of a toothpaste is provided which contains calcium, phosphate, and fluoride salts to provide remineralization in the oral cavity and as well includes sodium percarbonate so as to generate peroxide and, as well, sodium bicarbonate to provide abrasive action and a pleasant mouth feel after brushing in a composition which is stable prior to use, yields effective amounts of components in the oral cavity during use, and is quite palatable. In accordance with this invention, the remineralization salts of calcium and phosphate, as well as the sodium percarbonate and sodium bicarbonate, are present in an anhydrous vehicle whereas the fluoride salt is present in an aqueous-containing vehicle. Both vehicles are provided in a dispenser which dispenses the components simultaneously to an applicator such as a toothbrush for use in the oral cavity. It has been found that by maintaining the sodium percarbonate and sodium bicarbonate in an anhydrous vehicle, the adverse reaction between the same is avoided. Likewise, maintaining the calcium and phosphate salts in the anhydrous vehicle prevents premature reaction between the same and prevents reaction with the fluoride, which is in a separate aqueous-containing vehicle. Upon simultaneous application to the oral cavity, the anhydrous vehicle prevents immediate reaction between the calcium, phosphate, and fluoride salts to maintain an effective amount of free fluoride in the oral cavity to provide the effective remineralization activity. The aqueous-containing vehicle also provides the proper flowability and desired palatability needed for consumer use.

### DETAILED DESCRIPTION OF THE INVENTION

Sodium bicarbonate is incorporated in the dentifrice of the present invention in an amount of about 10 to 65%, preferably within the range of about 30 to 60%, by weight. For toothpaste formulations, preferably at least 30% of the sodium bicarbonate abrasive has particle sizes less than about 25 microns, and the median particle size is desirably less than 44 microns (Grade 3DF). Use of bicarbonate abrasive particles of this size range imparts an acceptable level of abrasivity and smooth consistency to the product. Coarser grades of baking soda could be used if desired for particular applications or when a more granular texture is desired. Coarser grades of bicarbonate are particularly desirable when clear or partially clear gels are desired (see commonly assigned U.S. Pat. No. 4,943,429 issued Jul. 24, 1990, entitled “Dentifrice Gels Containing Sodium Bicarbonate”).

The hydrogen peroxide-releasing agent, sodium percarbonate, is present in the dentifrice in an amount of about 1 to 10%, preferably within the range of about 3-7%, by weight. The sodium percarbonate ingredient of an invention dentifrice composition is employed in the form of a crystalline powder, which typically has an average particle size between about 1-100 microns, and preferably the particle size is in the range of 5-40 microns. Methods of manufacturing sodium percarbonate are described in technical publications such as U.S. Pat. No. 4,966,762 and references cited therein.

The products of this invention preferably contain from about 0.05% to about 15.0% by weight, more preferably from about 0.10% to about 10.0% by weight, of the calcium salt(s); from about 0.05% to about 15.0% by weight, more preferably from about 0.10% to about 10.0% by weight, of the phosphate salt(s); and from about 0.01 % to about 5.0%, more preferably from about 0.02% to about 2.0%, by weight of the fluoride salt(s).

The products of this invention contain a molar ratio of the calcium salt(s) to the phosphate salt(s) of preferably from about 0.01:1 to about 100:1. Most preferably, the concentration of the calcium salt(s) and the concentration of the phosphate salt(s) are preferably essentially the same in the products of this invention. The concentration of the calcium salt(s) always exceeds the solubility of such salt, whereas the concentration of the phosphate salt(s) may be as high as or even higher than the solubility thereof.

As a calcium compound, it is, in principle, possible to employ in the preparations of the invention any water-soluble or partially water-soluble toxicologically harmless calcium compound.

A compound is considered to be water-soluble when at least 0.25 gram thereof dissolves in 100 ml of H₂O at 20° C. Suitable water-soluble calcium compounds are, for example, calcium chloride, calcium bromide, calcium nitrate, calcium acetate, calcium gluconate, calcium benzoate, calcium glycerophosphate, calcium formate, calcium fumarate, calcium lactate, calcium butyrate and calcium isobutyrate, calcium malate, calcium maleate, calcium propionate, or mixtures of water-soluble calcium compounds. In the compositions of the invention for the remineralization of human dental enamel, at least about 100 ppm and preferably at least about 1000 ppm of calcium ions should be present; the upper limit is about 35,000 ppm of calcium ions.

As used herein, the term “partially water-soluble” with respect to the calcium salt component refers to a calcium salt having a solubility which is greater than that of dicalcium phosphate dihydrate in an aqueous solution having a pH of about 7.0 and a temperature of about 25° C. but which is less than that solubility which would release more than about 1400 ppm of calcium cations in such aqueous solution. In an aqueous solution having a pH of about 7.0 at a temperature of about 25° C., dicalcium phosphate dihydrate generally releases about 40 ppm of calcium cations. Thus, a partially water-soluble calcium salt which can be used in the present invention generally has a solubility such that the salt is capable of releasing more than about 40 ppm but no more than about 1400 ppm of calcium cations in an aqueous solution having a pH of about 7.0 at a temperature of about 25° C. Preferably, the partially water-soluble calcium salt(s) used in this invention has a solubility in such aqueous solution such that the salt(s) releases from about 100 ppm to no more than about 1400 ppm of calcium cations.

Non-limiting examples of calcium salts of partial water-solubility suitable for use in this invention include calcium sulfate, anhydrous calcium sulfate, calcium sulfate hemihydrate, calcium sulfate dihydrate, calcium malate, calcium tartrate, calcium malonate, calcium succinate, and mixtures of the foregoing. Calcium sulfate is preferred.

Suitable water-soluble inorganic phosphates within the scope of the present invention are, for example, alkali salts and ammonium salts of orthophosphoric acid, such as potassium, sodium or ammonium orthophosphate, monopotassium phosphate, dipotasium phosphate, tripotassium phosphate, monosodium phosphate, disodium phosphate and trisodium phosphate. The concentration of the phosphate ions is at least about 100 ppm, and preferably at least about 1000 ppm to 40,000 ppm. Solubility in water is defined as in the case of the calcium compounds.

While the length of time of contact between the dissolved calcium and phosphate salts and the tooth's surface is not critical, it is necessary for the length of time to be great enough to allow diffusion of the ions through the tooth's surface to the demineralized subsurface. It is submitted that at least ten seconds is required for this diffusion and preferably it should be greater than thirty seconds and even longer if possible. The desired extended time for such diffusion is a benefit accruing from the use of the divalent metal salts of this invention.

Upon use in the oral cavity with saliva any solution should have a pH of from about 4.0 to 7.0 and preferably between about 5.0 and 5.75 before and after the precipitation reaction, and be otherwise compatible in the oral environment. The ions must not combine prematurely in the solution to form a precipitate, but must be able to diffuse through the surface of the tooth to a demineralized subsurface area and be able to form an insoluble salt with ions of the other solution.

Suitable fluoride salts for use in the present invention include the alkali fluorides such as sodium, potassium, lithium or ammonium fluoride; tin fluoride; indium fluoride; zirconium fluoride; copper fluoride; nickel fluoride; palladium fluoride; fluorozirconates such as sodium, potassium or ammonium fluorozirconate or tin fluorozirconate; fluorosilicates; fluoroborates; and fluorostannites.

Organic fluorides, such as the known amine fluorides, are also suitable for use in the products of the present invention.

Water-soluble alkali metal monofluoro-phosphates such as sodium monofluorophosphate, lithium monofluorophosphate and potassium monofluorophosphate, (the sodium monofluorophosphate being preferred) may be employed. In addition, other water-soluble monofluorophosphate salts may be employed, including, for example, ammonium monofluorophosphate, aluminum monofluorophosphate, and the like.

In accordance with this invention, the bicarbonate and percarbonate and remineralizing calcium and phosphate salts are incorporated into an anhydrous carrier. The water-free environment prevents the normally reactive components from reacting during storage and delays and aids in controlling reaction of the components in the oral cavity so as to allow the individual components to be effectively active. The anhydrous carrier principally comprises a polyethylene glycol (PEG) humectant suitably incorporated in an amount of about 20 to 75% by weight of the dentifrice. The preferred polyethylene glycol humectants are those having molecular weights between about 200 and 600, e.g., polyethylene glycols sold as Carbowax 200, 300, 400 or 600. PEG-8, Carbowax 400, is particularly useful.

The anhydrous dentifrice of the invention preferably contains other conventional adjuvants in addition to sodium bicarbonate, sodium percarbonate, calcium and phosphate salts, and polyethylene glycol. Such ingredients may include thickeners, sweeteners, flavors, surfactants, additional abrasives, or other additives known in the art.

Thickeners which are useful for thickening the anhydrous pastes or gels include the solid polyethylene glycols having molecular weights above about 900, e.g., those sold as Carbowax 900, 1000, 1450, 3350, 4600 or 8000, and the inorganic amorphous silicas, or hydro-gels, including Tixosil 43 and Tixosil 63 from Kofran Chemical Co., Sylodent 15 or Sylodent 2 from W.R. Grace and Co., or the pyrogenic or fumed silicas such as Aerosil 200 from Degussa, or Cabosil from Cabot.

In general, it has been found that most other conventional organic gelling agents such as sodium CMC are not effective in thickening the dentifrice of the present invention because they require the presence of water to induce swelling. Organic thickeners which are soluble in polyethylene glycol and which do not require water to increase their viscosities in solution may be effective to impart a desirable viscosity and texture to the dentifrice hereof.

The inorganic amorphous silica thickeners may be incorporated in the anhydrous dentifrice of the invention in amounts of up to about 10%, preferably about 1.0 to 3.0% thereof. On the other hand, the organic thickeners may comprise up to about 5%, preferably about 0.5 to 2.0%, of the composition.

Suitable sweeteners may also be included in the dentifrice of the invention. Such sweeteners include sucrose, lactose, maltose, sorbitol, saccharin, sodium or calcium cyclamate, aspartame or other sweeteners known to those skilled in the art. The sweetener is desirably present within the range of from about 0.1 to 5.0%.

Flavoring agents useful in the dentifrice of the present invention include the flavoring oils, for example, oils of peppermint, spearmint, menthol, wintergreen, clove, sassafras, cinnamon, lemon, orange, licorice, sage, marjoram or eucalyptus, as well as sodium methyl salicylate. The flavoring agent may be present in the dentifrice in an amount of about 0.2 to 2.0% by weight of the dentifrice, preferably within the range of about 0.5 to 1.0%.

Suitable surfactants include water soluble anionic surfactants such as the sulfates of long chain (C₈ -C₁₈) alcohols, e.g., sodium lauryl sulfate or sodium tridecylsulfate; the sulfates or sulfonates of monoglycerides of fatty acids, e.g., sodium lauroyl glyceryl sulfate or sodium coconut monoglyceride sulfonate; the sulfonates of succinic esters, e.g., sodium dioctyl sulfosuccinate; the alkyl sulfoacetates such as sodium lauroyl sulfoacetate or sodium coconut sulfoacetate; the salts of sulfoacetic acid modified by aminoethyl long chain fatty acid esters such as sodium sulfocolaurate; the amides formed from higher fatty acids with short chain aliphatic amino acids such as sodium lauroyl sarcosinate or sodium methyl lauroyl tauride; and soaps such as the sodium, potassium or triethanolamine salts of fatty acids. Similarly, nonionic surfactants may be used such as the ethoxylated sugar esters of the higher fatty acids, for example, ethoxylated sorbitan monostearate and ethoxylated glycerol monostearate. Other nonionic surfactants include polyethylene glycol/polypropylene glycol copolymers, e.g., Pluraflo L1220 (BASF). Also, amphoteric surfactants such as the mono or dicarboxylated imidazoline derivatives of fatty acids, e.g., sodium lauryl dicarboxy imidazoline or sodium coconut dicarboxy imidazoline may be used. Cationic surfactants may also be used in the gel. Surfactants may be selected which additionally impart significant antibacterial action to the gel. Examples of such surfactants include benzyl dimethyl stearyl ammonium chloride and cetylpyridinium chloride.

The surfactant is incorporated in the dentifrice in an amount of about 0 to 8%, preferably within the range of about 0.2 to 6.0% of the dentifrice.

The dentifrice may also contain from about 0 to 40% by weight of an additional abrasive material or materials. Abrasive materials suitable as additional abrasives in the dentifrices of the present invention are well known in the art and include calcium carbonate, e.g., chalk; dicalcium phosphate dihydrate, anhydrous dicalcium phosphate, tricalcium phosphate, calcium pyrophosphate, sodium meta-phosphate; amorphous silica; alumina; titanium dioxide; zirconium silicate; and the like.

If a fluoride-containing agent is to be incorporated into the dentifrice of the present invention, the dentifrice needs to be divided into two parts, including a first part comprising an anhydrous composition containing sodium bicarbonate, sodium percarbonate, and the calcium and phosphate remineralization salts, and a second part formed of an aqueous-based composition containing the fluoride component such as those described previously. It has been found that by incorporating the fluoride in a carrier separate from the anhydrous carrier, reaction such as between the calcium and fluoride can be prevented during storage and controlled during use so as to prevent the rapid precipitation of calcium fluoride and consequent loss of free fluoride in the oral cavity. To aid in the remineralization process, sufficient free fluoride needs to be present in the oral cavity during use of the dentifrice. Separating the fluoride component in an aqueous-based system not only prevents the premature reaction of the components, but also provides a dentifrice component which can, when combined with the anhydrous composition, yield a readily-flowable composition which is palatable. The aqueous component of the dentifrice composition of this invention includes only the fluoride as the sole active component.

A humectant and water system is included as the carrier for the fluoride salt. Humectants are usually polyols which, for example, may include glycerol, sorbitol, propylene glycol, lactitol, xylitol, polypropylene glycol, polyethylene glycol, hydrogenated corn syrup and mixtures thereof. Generally the amount of humectant will range from about 25 to 90%, preferably from about 40 to 70% by weight. Particularly preferred is a liquid mixture of 3 to 30% water, 0 to 80% glycerol and/or 20 to 80% sorbitol. As in the anhydrous part, the aqueous part of the dentifrice can include adjuvants such as thickeners, sweeteners, flavors, surfactants, and abrasive or other additives known in the art.

A dentifrice in accordance with this invention, which contains the composition in two parts comprising a first part which comprises sodium bicarbonate, sodium percarbonate, and the calcium and phosphate remineralization salts in an anhydrous carrier, and a second part containing a fluoride salt in a water-humectant system, must be packaged for use in a container that physically divides the two parts, but which can dispense the two parts simultaneously. One such container is a squeezable tube which contains a physical divider therein which divides the tube into sections, but which allows both parts of the tube to be dispensed from the tube simultaneously. Such tubes and methods of making same are disclosed in commonly assigned U.S. Pat. Nos. 6,210,621; 5,860,565; and RE 36,035. Alternatively, the aqueous and anhydrous parts of the dentifrice of this invention can be stored and dispensed from a pump dispenser which, again, divides the aqueous and anhydrous parts during storage, but dispenses the components simultaneously from the container. Such pump dispensers are described in commonly assigned U.S. Pat. Nos. 5,020,694; 5,038,963; 5,645,193; and 5,289,949. All of these patents listed to provide separation and dispensing the dentifrice of this invention are herein incorporated by reference in their entirety.

The following Example illustrates preferred embodiments of the dentifrice of the invention. Unless otherwise noted, all parts and percentages specified above and given in the following Example are specified by weight of the complete dentifrice.

### EXAMPLE 1

| | A | B |
|---|---|---|
| | Component | Component |
| INGREDIENTS | % | % |
| CMC | 0.5000 | |
| SD Alcohol 38-B 190 Proof (denaturant | 1.3906 | |
| MeOH) | | |
| Sorbitol 70% soln | 61.5000 | |
| Water | 12.7719 | |
| FD&C Blue #1 | 0.0025 | |
| Sodium Fluoride | 0.5350 | |
| PEG-32 (PEG 1450 NF) | 2.0000 | |
| PEG-8 | | 33.0500 |
| Potassium Phosphate, Dibasic | | 1.8000 |
| Sodium Saccharin | 0.5000 | 0.8000 |
| Sucralose | | 0.2000 |
| Calcium Sulfate Anhydrous | | 4.0000 |
| PEG/PPG 116/66 copolymer | | 4.0000 |
| (Pluraflo L 1220) | | |
| Fumed Silica (Aerosil 200 VS) | | 2.5000 |
| Sodium Bicarbonate Gr. 3 USP low Fe | | 45.0000 |
| Thickening Silica (Tixosil 43) | 8.0000 | |
| Hydrated Silica (Tixosil 63) | 10.0000 | |
| Sodium Lauryl Sulfate | 1.5000 | 1.0000 |
| Mint Flavor | 1.3000 | |
| Mint Flavor | | 1.3500 |
| Sodium Carbonate Peroxide (Sodium | | 6.3000 |
| Percarbonate) | | |
| | 100.0000% | 100.0000% |

What is claimed is:

1. A dentifrice composition comprising a mixture of sodium bicarbonate, a peroxide generator, at least one water soluble or partially water soluble calcium salt, and at least one water soluble phosphate salt, said mixture contained within an anhydrous vehicle.

2. The composition of claim 1 wherein the peroxide generator is sodium percarbonate.

3. The composition of claim 2 containing 10-65% by weight sodium bicarbonate and 1-10% by weight sodium percarbonate.

4. The composition of claim 1 containing a partially water soluble calcium salt.

5. The composition of claim 4 wherein said partially water soluble calcium salt is calcium sulfate.

6. The composition of claim 4 containing 10-65% by weight sodium bicarbonate and 1-10% by weight sodium percarbonate.

7. The composition of claim 6 containing 30-60% by weight sodium bicarbonate and 3-7% by weight sodium percarbonate.

8. The composition of claim 1 wherein said vehicle comprises a polyethylene glycol humectant.

9. The composition of claim 8 wherein said polyethylene glycol humectant comprises from about 20-75% by weight of said composition.

10. The composition of claim 9 wherein said vehicle further contains a copolymer of polyethylene glycol and polypropylene glycol.

11. The composition of claim 9 comprising 10-65% by weight sodium bicarbonate and 1-10% by weight sodium percarbonate.

12. The composition of claim 11 containing a partially water soluble calcium salt.

13. The composition of claim 12 wherein said partially water soluble calcium salt is calcium sulfate.

14. The composition of claim 1 comprising 0.05-15% by weight each of said at least one calcium salt and phosphate salt.

15. The composition of claim 14 wherein said phosphate salt is an alkali metal phosphate.

16. The composition of claim 15 wherein said phosphate salt is dibasic potassium phosphate.

17. A dentifrice composition comprising a first part containing a mixture of sodium bicarbonate, a peroxide generator, at least one water soluble or partially water soluble calcium salt, and at least one water soluble phosphate salt, said mixture contained within an anhydrous vehicle, and a second part comprising a fluoride salt contained within an aqueous vehicle, said first part and second part being physically separated from each other.

18. The composition of claim 17 wherein said first and second parts are physically separated from each other in a dispenser which allows simultaneous dispensing of said first and second parts.

19. The composition of claim 17 wherein said fluoride salt is sodium fluoride.

20. The composition of claim 17 wherein the peroxide generator is sodium percarbonate.

21. The composition of claim 20 wherein said first part contains 10-65% by weight sodium bicarbonate and 1-10% by weight sodium percarbonate.

22. The composition of claim 17 wherein said first part contains a partially water soluble calcium salt.

23. The composition of claim 22 wherein said partially water soluble calcium salt is calcium sulfate.

24. The composition of claim 23 wherein said first part contains 10-65% by weight sodium bicarbonate and 1-10% by weight sodium percarbonate.

25. The composition of claim 24 wherein said first part contains 30-60% by weight sodium bicarbonate and 3-7% by weight sodium percarbonate.

26. The composition of claim 17 wherein said anhydrous vehicle comprises a polyethylene glycol humectant.

27. The composition of claim 26 wherein said polyethylene glycol humectant comprises from about 20-75% by weight of said first part of said composition.

28. The composition of claim 26 wherein said anhydrous vehicle further contains a copolymer of polyethylene glycol and polypropylene glycol.

29. The composition of claim 27 wherein said first part comprises 10-65% by weight sodium bicarbonate and 1-10% by weight sodium percarbonate.

30. The composition of claim 29 wherein said first part contains a partially water soluble calcium salt.

31. The composition of claim 30 wherein said partially water soluble calcium salt is calcium sulfate.

32. The composition of claim 17 wherein said first part comprises 0.05-15% by weight each of said at least one calcium salt and phosphate salt.

33. The composition of claim 32 wherein said phosphate salt is an alkali metal phosphate.

34. The composition of claim 33 wherein said phosphate salt is dibasic potassium phosphate.

35. The composition of claim 17 wherein said second part contains a humectant.

36. The composition of claim 35 wherein said second part contains 25-90% wt. % of said humectant and 3-30 wt. % water.
